(19) United States
(12) Patent Application Publication
(10) Pub. No.: US 2024/0091144 A1
(43) Pub. Date: Mar. 21, 2024

(54) MULTI-LAYER ORAL THIN FILM

(71) Applicant: LTS Lohmann Therapie-Systeme AG, Andernach (DE)

(72) Inventors: Michael LINN, Waldböckelheim (DE); Christoph SCHMITZ, Rheinbrohl (DE); Claudia NORELLI, Hillscheid (DE); Mario FICKER, Bonn (DE)

(21) Appl. No.: 18/272,441

(22) PCT Filed: Jan. 14, 2022
(86) PCT No.: PCT/EP2022/050800

§ 371 Date: Jul. 14, 2023

(30) Foreign Application Priority Data
Jan. 15, 2021 (DE) 10 2021 100 783.6

(51) Int. Cl. A61K 9/00; A61K 47/10; A61K 47/18

(52) U.S. Cl. CPC A61K 9/006; A61K 47/10; A61K 47/18

(57) ABSTRACT

The present disclosure relates to a multi-layer oral thin film comprising a first and a second matrix layer, which each contain at least one polymer, and a separation layer located between the first and the second matrix layer, wherein the separation layer comprises at least one polyethylene glycol, to a method for production of the oral thin film, and to the use thereof as a medicament.

The present invention relates to a multi-layer oral thin film, to a method for production thereof, and to the use of such an oral thin film as a medicament.

Oral thin films are thin films containing at least one pharmaceutically active agent that are placed directly in the oral cavity or against the oral mucosa and dissolve or macerate there and in so doing deliver the active agent. These films are, especially, thin, one- or multi-layer, active-agent-containing polymer-based films which, when applied to a mucous membrane, especially the oral mucosa, can deliver the active agent directly into same. The very good blood supply to the oral mucosa ensures a rapid transfer of the active agent into the bloodstream. This dosage system has the advantage that the active agent is absorbed for the most part by the mucous membrane, thus avoiding the first-pass effect, which occurs in the case of the conventional dosage form of an active agent in tablet form. The active agent may be dissolved, emulsified or dispersed in the film.

Multi-layer oral thin films can offer various advantages compared to “normal” one-layer oral thin films. For example, a high area density can be achieved by the combination of several layers, and various layers can be combined, such as an active agent layer with a pH-regulating layer, or an active agent layer with a protecting backing layer.

Multi-layer oral thin films can be produced in principle in various ways, such as by multiple coating or by the gluing together or laminating together or prefabricated layers.

Multiple coating has various disadvantages here. For example, the active agent is thermally stressed more heavily by multiple coating operations. Furthermore, such a bond between the layers is often not strong and may delaminate again. In addition, errors in the coating (area density) are reproduced in the following layers.

The joining together of a plurality of layers by means of gluing also has disadvantages. Known adhesive layers often have a high density of functional groups, such as —OH groups, which promote the migration of active agent and/or auxiliary substances, such as buffer salts, through the adhesive layer. In addition, known adhesive layers are often poorly water-soluble and thus demonstrate poor dissolution behaviour in the patient's mouth (poor mouthfeel). In addition, the use of large amounts of plasticisers (for example 20% glycerol) is often necessary in the known adhesive layers, although these may migrate into the other layers of the oral thin film and change there the physical properties (for example lower the glass transition temperature).

The aim of the present invention was to overcome the disadvantages known from the prior art. Especially, a multi-layer oral thin film is to be provided, in which the individual layers are firmly joined together, wherein the known adhesive layers and/or the coating on one another can be spared. Furthermore, a migration of substances between the individual layers is to be largely prevented. In addition, the multi-layer oral thin film is to be producible as easily and as cost-effectively as possible.

These aims have now been addressed by an oral thin film according to claim 1, i.e. by a multi-layer oral thin film comprising a first and a second matrix layer, which each contain at least one polymer, and a separation layer located between the first and the second matrix layer, wherein the separation layer comprises at least one polyethylene glycol.

One of the advantages of using such a multi-layer oral thin film is that polyethylene glycol films are well suited as separation layers/connection layers/adhesive layers in multi-layer oral thin films due to their smooth surface, their low melting point or glass transition temperature, their harmless toxicity profile and their water solubility. By heating and/or high pressure, polyethylene glycol adhesive layers are especially suitable for connecting two other layers to one another. For example, an active-agent-containing layer and a pH-regulating buffer layer can be connected to one another.

In principle, a plurality of laminates can also be combined with different active agents/auxiliary substances, to form a “sandwich OTF”.

Furthermore, the polyethylene glycol layer is suitable as a barrier layer that prevents or minimises the migration of active agents or auxiliary substances (for example buffer salts) between the individual layers.

The polyethylene glycol adhesive layer also requires no or only small amounts of auxiliary substances such as plasticisers (for example 2-3% glycerol) and thus reduces the risk of migration of the adhesive layer components into the other layers of the oral thin film.

In the present document, the word “comprising” can also mean “consisting of”.

Hereinafter, the terms “separation layer”, “connection layer” and “adhesive layer” are to be understood synonymously.

Polyethylene glycols (PEG) are compounds of the general formula:

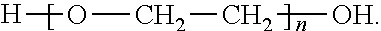

Higher molecular solid polyethylene glycols (melting temperature about 65° C.) are often also called polyethylene oxides or polyoxyethylenes (abbreviated to PEO or, more rarely, PEOX) or polywaxes. In this document, the terms “polyethylene glycol”, “polyethylene oxide” and “polyox” are used equivalently.

The following definitions in relation to the first and the second matrix layer always apply analogously for both the first and the second matrix layer.

In principle, the first and the second matrix layer can have the same composition or a different composition.

The multi-layer oral thin film according to the invention is preferably characterised in that the first and/or the second matrix layer comprises at least one water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble polymers preferably have a solubility in water of greater than 100 g/L at 25° C.

The at least one water-soluble polymer is preferably selected from the group consisting of starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums, wherein polyvinyl alcohols are especially preferred.

The multi-layer oral thin film according to the invention is preferably characterised in that the at least one polymer, preferably the water-soluble polymer, is provided in the particular matrix layer in an amount of from 10 to 90 wt. %, preferably from 20 to 60 wt. %, especially preferably from 30 to 50 wt. %, in relation to the total weight of the first and/or the second matrix layer.

The first and/or the second matrix layer preferably contains at least one pharmaceutically active agent.

The first and second matrix layer may contain the same or a different pharmaceutically active agent.

The at least one pharmaceutically active agent is in principle not subject to any restriction, but is preferably selected from all pharmaceutically active agents which are suitable for oral and/or transmucosal application.

According to the present invention, all pharmaceutically acceptable salts and solvates of the particular pharmaceutically active agent are also subsumed under the pharmaceutically active agent.

Preferred active agents are selected from the group comprising the active agent classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, although this group is not exhaustive.

The at least one pharmaceutically active agent is especially preferably ketamine and/or a pharmaceutically active salt or solvate thereof, preferably ketamine HCl.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyI)-2-(methylamino)cyclohexan-1-one.

(S)-ketamine or a pharmaceutically acceptable salt thereof, especially (S)-ketamine HCl, is especially preferably present as a single stereoisomer of ketamine, since the analgesic and anaesthetic potency of (S)-ketamine is approximately three times higher than that of the (R) form.

The active agent content in the first and/or the second matrix layer can vary within relatively wide limits. A range of from 10 to 60 wt. %, in relation to the dry weight of the particular matrix layer, can be stated as suitable. In one embodiment, the proportion of active agent in the particular matrix layer lies rather in the lower range, for example if the active agent has a strong unpleasant taste, which has to be compensated for by a greater amount of taste-masking agents. In this case, a range of from 10 to 40 wt. % can be stated as suitable active agent fraction. In another embodiment, the proportion of active agent in the dosage form according to the invention lies rather in the upper range, wherein a content of from 40 to 60 wt. % and especially a content of from 45 to 55 wt. % can be stated as being especially preferred.

(S)-ketamine or a pharmaceutically acceptable salt thereof is especially preferably present in the particular matrix layer in an amount of from 45 to 55 wt. % in relation to the dry weight of the first and/or the second matrix layer.

The multi-layer oral thin film according to the invention is further preferably characterised in that the first and/or the second matrix layer comprises at least one auxiliary substance selected from the group comprising colouring agents, flavourings, sweeteners, plasticisers, taste-masking agents, emulsifiers, enhancers, pH regulators, humectants, preservatives and/or antioxidants.

Each of these auxiliary substances is preferably contained in the particular matrix layer in an amount of from 0.1 to 40 wt. %, preferably from 0.1 to 30 wt. %, especially preferably from 0.1 to 15 wt. %, very especially preferably from 0.1 to 10 wt. %, or 0.1 to 5 wt. %, in relation to the total weight of the first and/or the second matrix layer.

The at least one polyethylene glycol preferably has a mean molecular weight of from at least 2,000 g/mol to 7,000,000 g/mol, or from 8,000 g/mol to 7,000,000 g/mol, preferably from 8,000 g/mol to 300,000 g/mol, especially preferably from 95,000 g/mol to 105,000 g/mol, especially of about 100,000 g/mol, or from 195,000 g/mol to 205,000 g/mol, especially of about 200,000 g/mol.

The molecular weight is derived from the rheology measurements described below.

The multi-layer oral thin film according to the invention is further preferably characterised in that the at least one polyethylene glycol has a viscosity of from 30 mPas to 50 mPas, or from 65 mPas to 115 mPas, measured at 25° C.

The viscosities stated refer in each case to a 5 wt. % solution of polyethylene glycol in water and are measured on a Brookfield viscometer, model RVF, with spindle no. 1 at 50 rpm and at a temperature of 25° C.

Polyethylene glycols which for example are known under the trade names POLYOX WSR N-10 or POLYOX WSR N-80 (Dow Chemical) are especially preferred.

Furthermore, the polyethylene glycols PEG 2000, PEG 4000, PEG 6000, PEG 8000, PEG 10000 or PEG 20000 can be used.

The multi-layer oral thin film according to the invention is further preferably characterised in that the at least one polyethylene glycol is contained in the at least one separation layer in an amount of from 60 to 100 wt. %, preferably in an amount of from 80 to 100 wt. %, in relation to the total weight of the at least one separation layer.

The multi-layer oral thin film according to the invention is further preferably characterised in that the at least one polyethylene glycol is contained in the at least one separation layer in an amount of from 65 to 100 wt. %, or 70 to 100 wt. %, or 85 to 100 wt. %, or 90 to 100 wt. %, or 95 to 100 wt. %, in relation to the total weight of the at least one separation layer.

The multi-layer oral thin film according to the invention is further preferably characterised in that the at least one polyethylene glycol is contained in the at least one separation layer in an amount of from 60 to 97.5 wt. %, or 65 to 97.5 wt. %, or 70 to 97.5 wt. %, or 80 to 97.5 wt. %, or 85 to 97.5 wt. %, or 90 to 97.5 wt. %, or 95 to 97.5 wt. %, in relation to the total weight of the at least one separation layer.

The multi-layer oral thin film according to the invention is further preferably characterised in that the at least one polyethylene glycol is contained in the at least one separation layer in an amount of from 60 to 97.5 wt. %, or 65 to 97.5 wt. %, or 70 to 97.5 wt. %, or 80 to 97.5 wt. %, or 85 to 97.5 wt. %, or 90 to 97.5 wt. %, or 95 to 97.5 wt. %, as well as additionally 2 to 2.5 wt. % of at least one plasticiser, preferably glycerol, in relation to the total weight of the at least one separation layer.

The multi-layer oral thin film according to the invention is preferably characterised in that the at least one separation layer comprises at least one auxiliary substance selected from the group comprising colouring agents, flavourings, sweeteners, taste-masking agents, emulsifiers, enhancers, pH regulators, humectants, preservatives and/or antioxidants.

Each of these auxiliary substances is preferably contained in the separation layer in an amount of from 0.1 to 10 wt. %, preferably from 0.1 to 5 wt. %, especially preferably from 0.1 to 2.5 wt. %, in relation to the total weight of this layer.

The multi-layer oral thin film according to the invention is preferably characterised in that the at least one separation layer contains at least one plasticiser, preferably glycerol, preferably in an amount of from 0.5 to 5 wt. %, especially preferably in an amount of from 2 to 2.5 wt. %, in relation to the total weight of the at least one separation layer.

The multi-layer oral thin film according to the invention is further preferably characterised in that the first and/or the second matrix layer contains at least one water-soluble polymer, preferably a polyvinyl alcohol and tris(hydroxymethyl)aminomethane (TRIS).

TRIS is an abbreviation for tris(hydroxymethyl)aminomethane (THAM), also called tromethamine, trometamol (INN) and also TRIS buffer. Chemically, it is a primary amine with three alcoholic hydroxy groups.

TRIS is used as a buffer substance for biochemical, molecular biological, microbiological and pharmaceutical purposes. With a pKs of 8.2 (at 20° C.), TRIS has a good buffering capacity between pH 7.2 to 9.0.

A matrix layer which contains at least one water-soluble polymer, preferably a polyvinyl alcohol, and tris(hydroxymethyl)aminomethane (TRIS) will also be referred to hereinafter as TRIS buffer layer or, if polyvinyl alcohol is used, as TRIS-PVA layer.

Especially, the water-soluble polymer in such a TRIS buffer layer comprises polyvinyl alcohol.

A layer of this kind has the advantage that TRIS can be incorporated in percentage and molar terms with the highest proportion of buffer/PVA. With other salts/buffers (phosphates, carbonates, citric acid, etc.), on the other hand, PVA clumps or precipitates.

The TRIS-PVA compositions can be foamed well and form a film of high optical homogeneity.

Polyvinyl alcohols (abbreviated PVA or PVAL, occasionally also PVOH) are polymers of the general structure

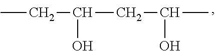

which in small proportions (about 2%) can also contain structural units of the type

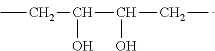

They belong to the group of vinyl polymers.

Commercially available polyvinyl alcohols, which are offered as white-yellowish powders or granular materials with degrees of polymerisation in the range of from about 500 to 2,500 (molar masses of about 20,000 to 100,000 g/mol), usually have degrees of hydrolysis of from 98 to 99 or 87 to 89 mol %, i.e. they still contain a residual content of acetyl groups. The polyvinyl alcohols are characterised by the manufacturers by specification of the degree of polymerisation of the starting polymer, the degree of hydrolysis, the saponification number and/or the solution viscosity.

According to the present invention, polyvinyl alcohols having a mean molecular weight of about 31,000 (PVA 4-88) to about 205,000 (PVA 40-88) g/mol are especially suitable in the TRIS buffer layer.

Furthermore, according to the present invention, polyvinyl alcohols with a viscosity of from 3.4-4.6 mPas (PVA 4-88) to 34-46 mPas (PVA 40-88) in a 40 g/l aqueous solution, determined by the falling ball method (Ph.Eur. 2.2.49), are especially suitable in the TRIS buffer layer, or mixtures of two or more different PVA types.

The oral thin film according to the invention is preferably characterised in that polyvinyl alcohol is contained in the TRIS buffer layer in an amount of from 20 to 90 wt. %, preferably from 40 to 80 wt. %, and very especially preferably from 50 to 75 wt. %, in relation to the total weight of the TRIS buffer layer.

The oral thin film according to the invention is further preferably characterised in that tris(hydroxymethyl)aminomethane is contained in the TRIS buffer layer in an amount of from 3 to 70 wt. %, preferably from 10 to 55 wt. %, and very especially preferably from 15 to 50 wt. %, in relation to the total weight of the TRIS buffer layer.

Preferably, the multi-layer oral thin film according to the invention, especially in the TRIS buffer layer, does not contain any other buffer substances other than TRIS, especially no phosphates, carbonates and/or citric acid.

Preferably no polymers other than PVA are contained in the TRIS buffer layer.

The multi-layer oral thin film according to the invention is preferably characterised in that the first and/or the second matrix layer is present in the form of a solidified foam that has voids.

The voids and the associated larger surface area of the films facilitate especially the access of water or saliva or other bodily fluids into the interior of the dosage form and thus accelerate the dissolution of the dosage form and the release of the active agent.

In the case of a rapidly absorbing active agent, transmucosal absorption can also be improved by the rapid dissolution of the matrix layer.

On the other hand, the wall thickness of said voids is preferably small, as these represent solidified bubbles, for example, so that rapid dissolution or destruction of these voids takes place.

A further advantage of this embodiment is that, despite the comparatively high area density, faster drying can be achieved by formulating as a foam than with a comparable non-foamed composition.

The multi-layer oral thin film according to the invention is preferably characterised in that the voids are isolated from one another and are preferably present in the form of bubbles, the voids being filled with air or a gas, preferably with an inert gas, especially preferably with nitrogen, carbon dioxide, helium or a mixture of at least two of these gases.

According to another embodiment, it is provided that the voids are connected to one another, preferably by forming a continuous channel system penetrating the matrix.

Said voids preferably have a volume fraction of from 5 to 98%, preferably from 50 to 80%, in relation to the total volume of the matrix layer. In this way, the advantageous effect of accelerating the dissolution of the matrix layer is influenced favourably.

Furthermore, surface-active agents or surfactants can be added to the matrix polymer or the polymer matrix for foam formation or to the obtained foam before or after drying in order to improve the stability of the foam before or after drying.

Another parameter that influences the properties of the dosage form according to the invention is the diameter of the voids or bubbles. The bubbles or voids are preferably created with the aid of a foam whipping machine, with which the diameter of the bubbles can be adjusted in a wide range, almost arbitrarily. Thus, the diameter of the bubbles or voids can be in the range of 0.01 to 350 μm. Especially preferably, the diameter is in the range of 10 and 200 μm.

The multi-layer oral thin film according to the invention is, in principle, not limited in the number of layers contained.

Embodiments in which the multi-layer oral thin film has further matrix layers in addition to the first and the second matrix layer are thus conceivable. The above definitions apply similarly for the further matrix layers. Similarly to the first and second matrix layers, these further matrix layers are connected to one another by an intermediate separation layer, as defined above.

In one embodiment, the multi-layer oral thin film comprises a first matrix layer, which contains ketamine, preferably (S)-ketamine, as pharmaceutically active agent, and a TRIS buffer layer as second matrix layer, which are connected to a separation layer, wherein the first and second matrix layer and separation layer are to be understood as defined above.

In one embodiment, the multi-layer oral thin film consists of a first matrix layer, which contains ketamine, preferably (S)-ketamine, as pharmaceutically active agent, and a TRIS buffer layer as second matrix layer, which are connected to a separation layer, wherein the first and second matrix layer and separation layer are to be understood as defined above.

The oral thin film according to the invention preferably has an area of from 0.5 cm² to 10 cm²,especially preferably from 2 cm² to 8 cm².

The oral thin film according to the invention is preferably characterised in that the area density of the multi-layer oral thin film is 10 to 500 g/m²,preferably 70 to 400 g/m².

The area density of the first matrix layer, the second matrix layer or any TRIS buffer layer that may be present and any other layer that may be present is in each case preferably at least 10 g/m², more preferably at least 20 g/m² or at least 30 g/m² or most preferably at least 50 g/m² or less than or equal to 400 g/m², more preferably less than or equal to 350 g/m² or less than or equal to 300 g/m² or most preferably less than 250 g/m². Preferably, the area density is 10 to 400 g/m², more preferably 20 to 350 g/m², or 30 to 300 g/m², most preferably 50 to 250 g/m².

Preferably, each of the provided layers has a layer thickness of preferably from 10 μm to 500 μm, especially preferably from 20 μm to 300 μm.

If the various layers are present in the form of a solidified foam, it is thus preferred that each of the layers present as a foam have a layer thickness of preferably 10 μm to 3,000 μm, especially of from 90 μm to 2,000 μm.

The oral thin film according to the invention can be produced according to methods known to a person skilled in the art.

The oral thin film according to the invention, however, is preferably produced by a method comprising the following steps:
a) producing and spreading a solution or suspension comprising the at least one polyethylene glycol, and then drying the spread solution or suspension in order to obtain a film comprising the at least one polyethylene glycol,
b) providing a first and a second matrix layer, which each comprise at least one polymer,
c) positioning the film obtained in step a) on the first matrix layer and positioning the second matrix layer on the film obtained in step a) so that the film obtained in step a) lies between the first and the second matrix layer in order to form a loose grouping, and
d) forming a firm bond by heating and/or by applying pressure to the loose grouping from step c).

In step d) heating is preferably performed to a temperature of from 30 to 200, preferably from 50 to 90.

In step d) a pressure of from 0.001 bar to 20 bar, preferably of from 0.01 bar to 8 bar is preferably applied.

The first and/or second matrix layer is preferably provided by a method comprising the following steps:
a1) producing a solution, dispersion or melt comprising at least one polymer,
aa1) optionally foaming the solution, dispersion or melt from step a1) by introducing a gas or gas mixture by chemical gas generation or by expansion of a dissolved gas,
b) spreading the solution, dispersion or melt from step a) or the optionally foamed solution, dispersion or melt from step aa1) in order to obtain a first and/or second matrix layer.

It is clear to a person skilled in the art that step aa1) is then only necessary if the first and/or the second matrix layer is to be present in the form of a solidified foam that has voids.

The present invention further relates to a multi-layer oral thin film obtainable by the method described above.

The present invention additionally relates to a multi-layer oral thin film as described above, or obtainable by the method described above, as a medicament.

The present invention additionally relates to a multi-layer oral thin film, as described above or obtainable by the method described above, wherein ketamine, preferably S-ketamine, or a pharmaceutically acceptable salt thereof, is used as pharmaceutically active agent in the first and/or the second matrix layer, for use in the treatment of pain and/or depression, especially to reduce the risk of suicide and/or for use as a general anaesthetic, preferably to initiate and carry out general anaesthesia, or as a supplement in the case of local anaesthesia and/or as an analgesic.

The preferred embodiments described above for the multi-layer oral thin film according to the invention are also applicable for the method according to the invention, the multi-layer oral thin film obtained by this method, and use thereof as a medicament.

The invention will be explained in greater detail hereinafter on the basis of non-limiting examples.

### EXAMPLES

### Example 1

### Production of a First Matrix Layer With Polyox Top Layer:

Example formulation formed of a polyox layer (not foamed) and an S-ketamine-containing first matrix layer (in the form of a foam) by multiple coating.

TABLE 1

| | 1 |
|---|---|
| Material | [wt. %] |
| (S)-ketamine HCl | 50.00% |
| PVA 4-88 | 41.70% |
| PVA 40-88 | — |
| Saccharin Na | 1.00% |
| Sucralose | 2.00% |
| Glycerol | 2.30% |
| Flavouring 1 | 1.00% |
| Flavouring 2 | 2.00% |
| Polyox WSR N10 | 97.50% |
| Glycerol | 2.3% |
| FD&C Red 40 (colouring agent) | 0.20% |
| Solvent | Purified water |
| Area density (dry) | 1^{st} matrix layer: |
| | 118.7 g/cm² |
| | Separation layer: |
| | 100 g/cm² |
| Coating weight (incl. | 1^{st} matrix layer: |
| res. water) | 121.9 g/cm² |
| | separation layer |
| | 101.5 g/cm² |

### Production:

Formulation 1 is a two-layer formulation, wherein a first polyox layer forms an active-agent-free separation layer which is produced first. This layer is then coated with an active-agent-containing matrix layer, wherein 50% (S)-ketamine HCl is contained in a polyvinyl alcohol (PVA) 4-88 foam matrix. The dry area density for the polyox separation layer is defined with 100.0 g/m², which leads to a coating weight of 101.5 g/m² incl. 1.5% residual water. The dry area density for the active-agent-containing matrix layer is defined as 118.7 g/m², which leads to a coating weight of 123.6 g/m² incl. 4% residual water (expected value).

### Example 2

Example formulation formed of a polyox separation layer (not foamed) and an S-ketamine-containing first matrix layer (foamed) and a TRIS buffer layer as second matrix layer, connected by lamination:

TABLE 2

| | 2 |
|---|---|
| S-ketamine-containing 1^{st} matrix layer | [wt. %] |
| (S)-ketamine HCl | 50.00% |
| PVA 4-88 | 41.70% |
| Saccharin Na | 1.00% |
| Sucralose | 2.00% |
| Glycerol | 2.30% |
| Flavouring 1 | 1.00% |
| Flavouring 2 | 2.00% |
| Solvent | Purified water |
| Area density (dry) | 117.3 g/m² |

| | 3 |
|---|---|
| Polyox separation layer | [wt. %] |
| Polyox WSR N10 | 97.50% |
| Glycerol | 2.3% |
| FD&C Red 40 (colouring agent) | 0.20% |
| Solvent | Purified water |
| Area density (dry) | 98.2 g/m² |

| 2^{nd} matrix layer | 4 |
|---|---|
| (TRIS buffer layer) | [wt. %] |
| PVA 4-88 | 36.15 |
| PVA 40-88 | 36.15 |
| TRIS | 20.00 |
| Saccharin Na | 1.00 |
| Sucralose | 2.00 |
| Glycerol | 4.50 |
| FD&C Red No. 40 (colouring agent) | 0.20 |
| Solvent | Purified water |
| Area density (dry) | 192.8 g/m² |
| Coating weight (incl. res. water) | 200.6 g/m² |

The three layers were produced separately and the obtained laminates were heated to 70° C. and connected by lamination. After cooling, the laminates were firmly connected.

### pH Measurement

The pH value of the individual layers from Table 2 and also of the composites formed from these layers was measured.

TABLE 3

| | Temperature | |
|---|---|---|
| Sample | Medium | pH |
| TRIS layer 4 | 33.3° C. | 8.54 |
| | 2 mL water | |
| Kea layer 2 | 33.4° C. | 5.71 |
| | 2 mL water | |
| Polyox layer 3 | 33.3° C. | 7.58 |
| | 2 mL water | |
| TRIS/Polyox/Kea | 33.6° C. | 7.24 |
| (3-layer OTF) | 2 mL water | |
| TRIS layer 4 | 30.3° C. | 8.48 |
| | 2 mL glandosane¹ | |
| Kea layer 2 | 33.7° C. | 5.46 |
| | 2 mL glandosane¹ | |
| Polyox layer 3 | 32.6 | 6.02 |
| | 2 mL glandosane¹ | |
| TRIS/Polyox/Kea | 31.8° C. | 7.06 |
| (3-layer OTF) | 2 mL glandosane¹ | |

¹glandosane = artificial saliva

### Example 3

The migration of substances between individual layers of a multi-layer oral thin film was examined, wherein the individual layers are connected to one another by various methods.
1. Two-layer oral thin film (OTF1)—connection of the layers by a seam.

The two layers of OTF1 have a composition according to the following Tables 4 and 5, respectively.

TABLE 4

| Material | [wt. %] |
|---|---|
| PVA 4-88 | 39.50 |
| Saccharin Na | 1.0 |
| Sucralose | 2.0 |
| Raspberry flavour | 2.0 |
| Masking flavour | 1.0 |
| Glycerol | 4.5 |
| S-ketamine HCl | 50.0 |
| Area density | 121.9 g/m² |

TABLE 5

| Material | [wt. %] |
|---|---|
| PVA 4-88 | 36.15 |
| PVA 40-88 | 36.15 |
| TRIS | 20.00 |
| Saccharin Na | 1.00 |
| Sucralose | 2.00 |
| Glycerol | 4.50 |
| FD&C Red No. 40 | 0.20 |
| Area density | 200.6 g/m² |

Two individual layers with a composition according to Tables 4 and 5, respectively, and with an area of 2.72 cm² were produced and sewn by hand using a PVA thread of 0.2 mm thickness (Vis Extrusion GmbH, Hohberg, Germany).
2. Two-layer oral thin film (OTF2)—connection of the layers by sealing.

The two layers of OTF2 have a composition according to Tables 4 and 5 above, respectively.

Two individual layers with a composition according to Tables 4 and 5, respectively, and an area of 2.72 cm² were produced and connected by sealing. To this end, a hand-held sealing apparatus (Polystar 100 GEW at level 9 and with 850 μm feeler gauge distance) was used.
3. Three-layer oral thin film (OTF3)—connection of the layers by a polyox intermediate layer.

The three layers of OTF3 have a composition according to the above Table 5 and the following Tables 6 and 7.

TABLE 6

| | Form formulation |
|---|---|
| Material | [wt. %] |
| PVA 4-88 | 41.70 |
| Saccharin Na | 1.00 |
| Sucralose | 2.00 |
| Glycerol | 2.30 |
| Masking Flavour PHL-131985 | 1.00 |
| Raspberry Flavour PHL-097336 | 2.00 |
| S-ketamine HCl | 50.00 |
| Area density | 121.9 g/m² |

TABLE 7

| Material | [wt. %] |
|---|---|
| Polyethylene oxide MW 100,000 | 97.50 |
| Glycerol | 2.30 |
| FD&C Red No. 40 | 0.20 |
| Area density | 101.5 g/m² |

A three-layer oral thin film was produced, wherein the three layers have a composition according to the above Table 5 and Tables 6 and 7.

To this end, the individual layers were arranged in the form of a loose grouping, so that the polyox layer (Table 7) was in the middle. By heating the loose grouping to approximately 70° C., the polyox starts to melt and the layers of the loose grouping bond to one another.

The OTFs of all three formulations were sealed in COC bags of 60×60 mm and were stored in a 40° C. laboratory cabinet. The migration tests were planned for after 3 and 6 weeks of storage with n=3. Due to a misunderstanding, all 6 OTFs of the three formulations were removed from storage after 3 weeks, but only 3 OTFs were analysed. It was therefore possible to return the remaining 3 OTFs to the 40° C. cabinet after 3 weeks storage at room temperature. After a further 3 weeks at 40° C., the 6-week test point (3 weeks at 40° C.+3 weeks RT+3 weeks 40° C.) was analysed.

The migration of substances between the individual layers was examined.

TABLE 8
pH values (n = 3) in the unstored active-agent-containing layers.

| | | Not stored | | | |
|---|---|---|---|---|---|
| | | Sample 1 | Sample 2 | Sample 3 | Ø |
| OTF1 | sewn | 5.75 | 5.75 | 5.75 | 5.75 |
| OTF2 | sealed | 5.75 | 5.75 | 5.75 | 5.75 |
| OTF3 | polyox | 6.12 | 6.13 | 6.14 | 6.13 |

TABLE 9
pH values (n = 3) in the active-agent-containing layers.

| | | 3 weeks | | | | 6 weeks | | | |
|---|---|---|---|---|---|---|---|---|---|
| | | Sam- | Sam- | Sam- | | Sam- | Sam- | Sam- | |
| | | ple 1 | ple 2 | ple 3 | Ø | ple 1 | ple 2 | ple 3 | Ø |
| OTF1 | sewn | 6.47 | 6.43 | 6.48 | 6.46 | 6.53 | 6.46 | 6.45 | 6.48 |
| OTF2 | sealed | 6.44 | 6.54* | 6.54* | 6.51 | 6.44* | 6.42* | 6.45* | 6.44 |
| OTF3 | polyox | 6.23 | 6.19 | 6.18 | 6.20 | 6.32 | 6.29 | 6.28 | 6.30 |

*seal seam discarded

TABLE 10
pH value differences in the active-agent-containing layers

| | | Storage | | |
|---|---|---|---|---|
| | | Not stored | 3 weeks | 6 weeks |
| OTF1 | sewn | — | +0.71 | +0.73 |
| OTF2 | sealed | — | +0.76* | +0.69* |
| OTF3 | polyox | — | +0.07 | +0.17 |

*seal seam discarded

TABLE 11
Active agent content in the buffer-containing layers

| | | 3 weeks | | | 6 weeks | | |
|---|---|---|---|---|---|---|---|
| | | Content | % | | Content | % | |
| | | mg | LC | wt. % | mg | LC | wt. % |
| OTF1 | sewn | 1.0 | 6.2 | 1.9 | 1.0 | 6.1 | 1.8 |
| OTF2 | sealed | 0.9 | 5.3 | 2.0 | 0.4 | 2.7 | 1.1 |
| OTF3 | polyox | 0.6 | 3.8 | 1.0 | 0.8 | 4.8 | 1.4 |

LC = label claim 16.1 mg/OTF
n = 3

The lowest pH values and active agent contents were found in the polyox formulation. Formulation OTF3 thus appears to be the best possibility for combining the two layers with a lower migration of active agent.

1. A multi-layer oral thin film comprising a first and a second matrix layer, which each contain at least one polymer, and a separation layer located between the first and the second matrix layer, wherein the separation layer comprises at least one polyethylene glycol with a molecular weight from 8,000 g/mol to 7,000,000 g/mol in an amount of from 60 to 100 wt. %.

2. The multi-layer oral thin film according to claim 1, wherein the first and/or the second matrix layer comprises at least one water-soluble polymer.

3. The multi-layer oral thin film according to claim 2, wherein the at least one water-soluble polymer is selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

4. The multi-layer oral thin film according to claim 1, wherein the first and/or the second matrix layer comprise at least one pharmaceutically active agent, wherein the at least one pharmaceutically active agent is preferably selected from the group comprising the active agent classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, wherein the at least one pharmaceutically active agent preferably comprises ketamine, especially preferably (S)-ketamine.

5. The multi-layer oral thin film according to claim 1, wherein the first and/or the second matrix layer further comprises at least one auxiliary substance selected from the group comprising colouring agents, flavourings, sweeteners, plasticisers, taste-masking agents, emulsifiers, enhancers, pH regulators, humectants, preservatives and/or antioxidants.

6. The multi-layer oral thin film according to claim 1, wherein the at least one polyethylene glycol has a mean molecular weight of from 8,000 g/mol to 300,000 g/mol.

7. The multi-layer oral thin film according to claim 1, wherein the at least one polyethylene glycol has a viscosity of from 30 mPa s to 50 mPa s, measured in 5 wt. % aqueous solution at 25° C.

8. The multi-layer oral thin film according to claim 1, wherein the at least one polyethylene glycol is contained in the at least one separation layer in an amount of from 80 to 100 wt. %, in relation to the total weight of the at least one separation layer.

9. The multi-layer oral thin film according to claim 1, wherein the first and/or the second matrix layer contains at least one water-soluble polymer, preferably a polyvinyl alcohol and tris(hydroxymethyl)aminomethane.

10. The multi-layer oral thin film according to claim 9, wherein the water-soluble polymer, preferably the polyvinyl alcohol, is contained in the first and/or second matrix layer in an amount of from 20 to 90 wt. %, in relation to the total weight of this layer.

11. The multi-layer oral thin film according to claim 9, wherein tris(hydroxymethyl)aminomethane is contained in the first and/or second matrix layer in an amount of from 3 to 70 wt. %, in relation to the total weight of this layer.

12. The multi-layer oral thin film according to claim 1, wherein the first and/or the second matrix layer is present in the form of a solidified foam that has voids.

13. The multi-layer oral thin film according to claim 12, wherein the voids are isolated from one another and are preferably present in the form of bubbles, wherein the voids are filled with air or a gas, preferably with an inert gas, especially preferably with nitrogen, carbon dioxide, helium or a mixture of at least two of these gases.

14. The multi-layer oral thin film according to claim 12, wherein the voids are connected to one another and preferably form a channel system penetrating the particular matrix layer.

15. The multi-layer oral thin film according to claim 12, wherein the voids in the particular matrix layer have for a volume fraction of from 5 to 98%, preferably from 50 to 80%, in relation to the total volume of the particular matrix layer.

16. A method for producing a multi-layer oral thin film according to claim 1, comprising the steps of:
a) producing and spreading a solution or suspension comprising the at least one polyethylene glycol, and then drying the spread solution or suspension in order to obtain a film comprising the at least one polyethylene glycol,
b) providing a first and a second matrix layer, which each comprise at least one polymer;
c) positioning the film obtained in step a) on the first matrix layer and positioning the second matrix layer on the film obtained in step a) so that the film obtained in step a) lies between the first and the second matrix layer in order to form a loose grouping, and
d) forming a firm bond by heating and/or by applying pressure to the loose grouping from step c).

17. The method according to claim 16, wherein the first and/or second matrix layer is provided by a method comprising the following steps:
a1) producing a solution, dispersion or melt comprising at least one polymer,
aa1) optionally foaming the solution, dispersion or melt from step a1) by introducing a gas or gas mixture by chemical gas generation or by expansion of a dissolved gas,
b) spreading the solution, dispersion or melt from step a) or the optionally foamed solution, dispersion or melt from step aa1) in order to obtain a first and/or second matrix layer.

18. (canceled)

19. A method of administering a medicament comprising providing the multilayer oral thin film of claim 1 to a subject.

20. The multi-layer oral thin film according to claim 1, wherein the at least one polyethylene glycol has a molecular weight of about 100,000 g/mol, or of about 200,000 g/mol.

21. The multi-layer oral thin film according to claim 1, wherein the at least one polyethylene glycol has a molecular weight of from 95,000 g/mol to 105,000 g/mol, or from 195,000 g/mol to 205,000 g/mol.
